(19) United States
(12) Patent Application Publication
(10) Pub. No.: US 2008/0053323 A1
(43) Pub. Date: Mar. 6, 2008

(54) Method and apparatus for simultaneously applying two-color markings on an article

(75) Inventor: Karl L. Karlosky, Ocala, FL (US)

Correspondence Address: DAYCO PRODUCTS, LLC, 1 PRESTIGE PLACE, MIAMISBURG, OH, 45342, US

(73) Assignee: Dayoo Products, LLC

(21) Appl. No.: 11/513,372

(22) Filed: Aug. 30, 2006

(51) Int. Cl. A01H 5/02

(52) U.S. Cl. 101/171

(57) ABSTRACT

An apparatus and method for simultaneously or sequentially applying a plurality of distinctive markings onto the surface of an article such as a flexible polymeric hose is described in which the marking apparatus comprises a plurality of reservoirs having a plurality of marking fluids stored therein, wherein each reservoir has a marking fluid stored therein having a color which is distinctive from the color of any other marking fluid stored in any other reservoir; and a plurality of initiators operably associated with the plurality of reservoirs for initiating flow of the marking fluid from the reservoirs; a plurality of conduits for transporting the marking fluid from the reservoirs to an applicator device wherein the applicator device comprising a plurality of marking members for applying the marking fluid onto the article,

### BACKGROUND OF THE INVENTION

The invention relates to a method and apparatus for marking articles of manufacture on an assembly line and, more particularly, to a method and apparatus for placing multiple color markings, such as identification markings at spaced intervals along the length of a tubular structure or hose during the finishing operation.

It is desired and sometimes required that various articles such as hoses, tubes and cables carry identification markings at spaced intervals along their length. Typically, such markings are in the form of lines, circles, text, etc., as indicators for the assembly location. Normally, markings are in a color which contrasts that of the hose and layline. Typically, transfer stampers are employed wherein the markings are stamped directly on the outer surface of the article. Such transfer stampers are generally affixed into a check fixture to assure proper location and rotation of the marking. For special applications multiple markings may be desired so that various additional processes can be performed. If the markings are to be close together, it is impossible for a single stamper to make multiple stamps on the hose in one step, requiring the use of additional fixtures and/or stampers.

Accordingly, there is a need in the industry for an apparatus and method which allows for the simultaneous or sequential marking of a hose or cable with multiple markings which may be in the form of a plurality of colors or distinctive characteristics.

### SUMMARY OF THE INVENTION

The invention relates to an improved apparatus and method for simultaneously or sequentially marking an article with a plurality of distinct patterns. In particular, the invention relates to such as an apparatus and method for simultaneously or sequentially marking a flexible hose during continuous forming of the hose. In accordance with the invention, an apparatus for applying the multiple markings to the hose based upon predetermined locations for applying the markings are described. More particularly, the apparatus comprises a marking device containing a marking head or marking plate modified to simultaneously or sequentially apply a plurality of distinctive marks onto the surface of an article to be marked.

In one embodiment of the invention, the marking device is a marking head containing a plurality of nozzles through which a minute volume of marking fluid is forcefully ejected onto the surface of an article such as an extruded hose.

In another embodiment of the invention, the marking device is a marking plate having incorporated thereon a predetermined pattern image, the image of which is to be transferred to the surface of the article by gravity feed of the marking fluid.

A method for employing each of the above embodiment in applying a distinctive pattern to an article is also described.

### BRIEF DESCRIPTION OF THE DRAWINGS

FIG. 1 is a side illustration of a prior art marking system;

FIG. 2 is a perspective illustration of a first embodiment of a printing system of the present invention for marking a hose;

FIG. 3 is a top view illustration of the printing system of FIG. 2;

FIG. 4 is a perspective illustration of a second embodiment of a printing system of the present invention for marking a hose;

FIG. 5 is a top view illustration of the printing system of FIG. 4.

### DETAILED DESCRIPTION OF THE INVENTION

Hoses regularly incorporate markings of various types such as lines, circles, dots, text, geometric pattern, etc. as indicators for the assembly location. Typically, the markings are in contrasting colors to that of the hose. In special application, it may be desirable to apply two or more markings to the hose in which certain of the markings are to exhibit colors and/or patterns which are different from other markings applied to the hose. In applications where the markings are to be placed close together on the surface of the hose, it is impossible to mark the hose in a single step, particularly since the hose, in some cases, is marked as it is being formed. Therefore, prior to the present invention, multiple marking of hoses in which the markings are in close relationship to each other, it was necessary to apply such markings by employing multiple steps. Such multiple steps are both costly and time consuming, and are therefore highly undesirable from a manufacturing stand point.

According to the present invention an improved marking system which includes a modified marking device and method are used to apply a plurality of markings which may vary in color and/or design simultaneously to an article such as a hose in a single step, thereby overcoming the disadvantages of the prior systems. If desired, such markings may be applied sequentially depending on the particular marking or location of the marking that is to be applied.

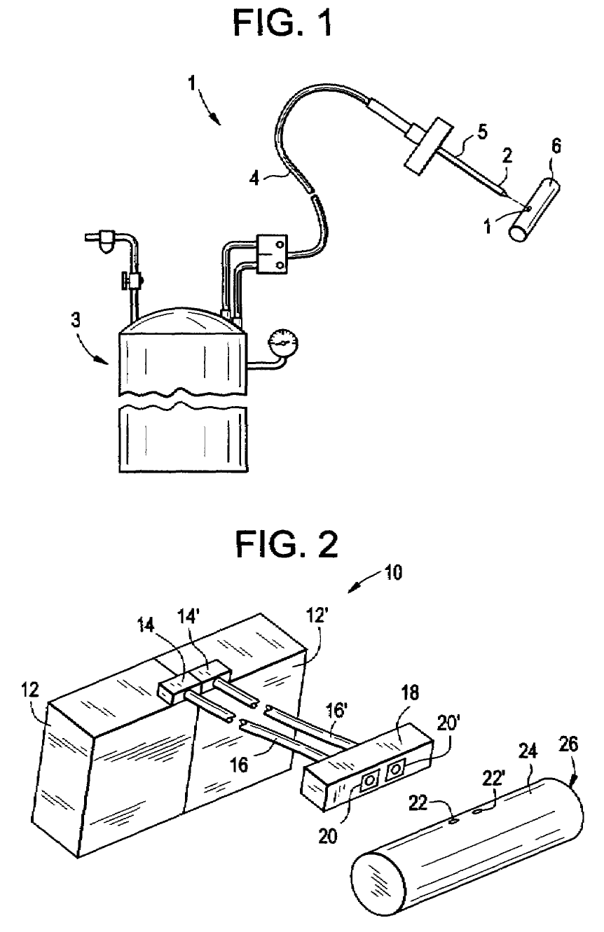

FIG. 1 illustrates a prior art marking system 1 wherein the marking fluid 2 is supplied from a pressured reservoir 3 through a conduit 4 to a single nozzle 5 where it is ejected onto a surface of a hose 6 to provide a desired marking. Such marking system marks the hose with a single definitive mark 7, but when multiple marks are desired requiring different color markings or if the marking locations are close together, it is impossible to mark the hose in a single step requiring significant down time and additional features to complete the markings. Such down time and additional features are undesirable from a manufacturing stand point. Therefore, it is an object of the invention to provide a novel printing apparatus for supplying multiple colors of marking fluids and simultaneously placing such markings on the surface of an article such as a hose. It is another object of the invention to provide a novel printing head for simultaneously placing a plurality of markings on an article such as a polymeric hose. It is still another object of the invention to provide a method for simultaneously marking an article such as a polymeric hose with a plurality of marking fluids using the novel printing apparatus and novel printing head. It is yet another object of the invention to reduce manufacturing costs and down time in the printing of multiple markings on an object.

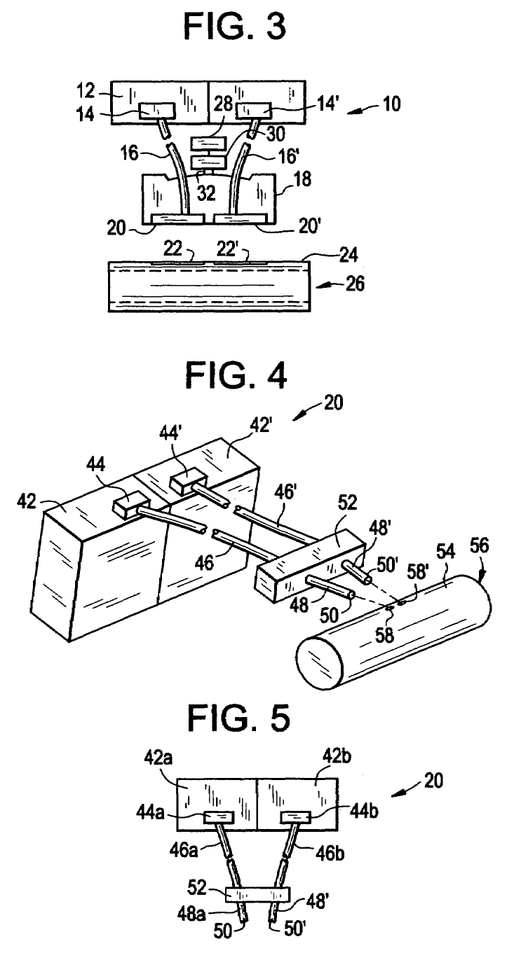

FIGS. 2 through 5 illustrate printing apparatuses for providing multiple markings on an article such as a flexible hose during the formation of the hose. FIGS. 2 and 3 illustrate a first embodiment of the invention wherein printing apparatus 10 comprises a first reservoir 12 containing a first marking fluid and a second reservoir 12′ containing a second marking fluid. The first reservoir 12 and the second reservoir 12′ may contain marking fluids of the same color or such fluids may be different colors. In accordance with the first embodiment of the invention, the first marking fluid is withdrawn from reservoir 12 by first initiator 14 and forced through first transport tube 16 to a stamping head 18 where the first marking fluid is ejected onto first marking head 20. Simultaneously therewith, second marking fluid P is withdrawn from a second reservoir 12′ by second initiator 14′ and forced through second transport tube 16′ to a stamping head 18 where the second marking fluid P is ejected onto second marking head 20′. At a predetermined instance, the stamping head 18 is activated by an activating device 30 (FIG. 3) which forces the stamping head 18 including the first and second marking heads 20 and 20′ to be urged forward with a swift and precise stamping action wherein the desired markings 22 and 22′ are produced on the outer surface 24 of the hose 26. The stamping head 18 is secured to the printing system by securing a securing device 28 (FIG. 3).

FIGS. 4 and 5 illustrate a second embodiment of the invention wherein printing apparatus 20 comprises a first reservoir 42 containing a first marking fluid and a second reservoir 42′ containing a first marking fluid As with the first embodiment of the invention, the marking fluids in the two reservoirs may be of the same color or such fluids may be different colors. In accordance with the second embodiment of the invention, the first marking fluid is withdrawn from reservoir 42 by initiator 44 and forced through transport tube 46 to a nozzle 48 where marking fluid is ejected through nozzle tip 50 directly onto the outer surface 54 of hose 56. Simultaneously therewith, second marking fluid is withdrawn from reservoir 42′ by initiator 44′ and forced through transport tube 46′ to a nozzle 48′ where minute volumes of the marking fluids and are ejected through nozzle tip 50′ directly onto the outer surface 54 of hose 56 to define the markings 58 and 58′ thereon at each predetermined location. The nozzles 48 and 48′ are maintained in a spaced relation from the surface of the hose by a distance D which, broadly, may be in the order of a few mm to several mm or more.

The marking fluid may be delivered from the reservoirs 12 and 12′ to the marking heads 20 and 20′ in the first embodiment, or to the nozzle tips 50 and 50 in the second embodiment, by pneumatic pressure or by gravity. Preferably, the marking fluid is delivered by pneumatic pressure The initiators 14 and 14′ of the first embodiment and the initiators 44 and 44′ of the second embodiment are activated by an activating means which contains predetermined instructions for delivering the desired amount of marking fluids in the desired increments at the desired time to produce the precise markings on the article. Any of the known devices for creating fluid flow pulsations can be used in the present invention for delivering the marking fluids in a pulsating manner. Typically, the pulsations of each marking fluid is created by a solenoid or piezo initiator. Typically, a solenoid is a cylindrical coil of insulated wire in which an axial magnetic field is established by a flow of electric current. The solenoid acts as a switch which may be opened and closed rapidly to cause pulsations of the marking fluid. Piezo initiators are non-conducting ceramic crystals that exhibit piezoelectric properties when they become mechanically strained. A piezo crystal is somewhat like a flexible loudspeaker cone which flexes when an electric current is applied.

The marking fluid employed in the present invention may be any suitable marking liquid which dries, or is made to dry, upon being exposed to ambient air. If desired, the drying of the marking fluid may be accelerated by the use of drying means directed to the markings as they are applied to the hose

The present invention is not meant to be limited to the specific method for applying a plurality of markings to an article or to the marking apparatus for applying the plurality of markings to the article described and illustrated herein, but may comprise any number of apparatuses and any method used to provide any number of individual and distinctive markings to the outer surface of a hose.

Although the invention has been described and exemplified herein with respect to the preferred embodiments thereof, it is to be understood that the invention is not limited to such embodiments, and that variations and modifications can be made therein without departing from the spirit and the scope of the invention defined by the appended claims.

What is claimed is:

1. An apparatus for simultaneously or sequentially applying a plurality of distinctive: markings onto the surface of an article in a single operation during the manufacture of said article, wherein said plurality of distinctive markings represent indicators for assembly location, said apparatus comprising:
a plurality of reservoirs having a plurality of marking fluids stored therein, wherein each reservoir of said plurality of reservoirs has stored therein a single marking fluid which is distinctive from any other color stored in any other reservoir of said plurality of reservoirs;
a plurality of initiators operably associated with said plurality of reservoirs for initiating fluid flow pulsations of said marking fluid from said reservoirs, wherein each initiator of said plurality of initiators is operably associated with a single reservoir of said plurality of reservoirs;
a plurality of conduits operably associated with said plurality of initiators, wherein each conduit of said plurality of conduits is operably associated with a single initiator of said plurality of reservoirs; and
an applicator device comprising a plurality of marking members for applying said marking fluid onto said article, said plurality of marking members operably associated with said plurality of conduits, wherein each of said marking members is operably associated with a single conduit of said plurality of conduits, wherein each nozzle of said plurality of nozzles is operably associated with a single conduit of said plurality of conduits.

2. The apparatus of claim 1 wherein said article is a flexible polymeric hose.

3. The apparatus of claim 1 wherein said plurality of distinctive markings is a plurality of one or more distinctive colors and/or distinctive patterns.

4. The apparatus of claim 1 wherein the flow of marking fluid through said plurality of initators is effected by pneumatic pressuree.

5. The apparatus of claim 1 wherein the flow of said marking fluid through said plurality of initators is effected by gravity.

6. The apparatus of claim 5 wherein said plurality of distinctive patterns are selected from the group consisting of numerals, geometrical designs and text.

7. The apparatus of claim 1 wherein each reservoir of said plurality of reservoirs contains a supply of distinctive colors therein for marking an article.

8. The apparatus of claim 1 wherein said applicator device is a stamping head comprising:
a plurality of marking heads, each of which is operably associated with a single conduit, each of said marking heads having an orifice through which said marking fluid flows from said associated reservoir through said associated conduit to said marking head upon activation of said initiator;
a securing device for securing said stamping head to said apparatus; and
an activator for forcing said stamping head to impinge upon the outer surface of an article for stamping a marking onto the surface of the article to be marked.

9. The apparatus of claim 1 wherein said applicator device is a printing head comprising:
a plurality of nozzles, each of which is associated with a single conduit through which said marking fluid flows from said associated reservoir through said associated conduit to said nozzle upon activation of said initiator;
a securing device for securing said plurality of nozzles to said apparatus; and
a plurality of nozzle tips, each of said plurality of nozzle tips having an orifice therein for ejecting minute volumes of marking fluid onto the surface of the article to be marked.

10. A method for simultaneously or sequentially applying a plurality of distinctive markings onto the surface of an article in a single operation during the manufacture of said article, wherein said plurality of distinctive markings represent indicators for assembly location, said method comprising:
forming an article;
placing a marking apparatus in close proximity to said article being formed, said marking apparatus comprising: a plurality of reservoirs having a plurality of marking fluids stored therein, wherein each reservoir of said plurality of reservoirs has stored therein a single marking fluid which is distinctive from any other color stored in any other reservoir of said plurality of reservoirs; a plurality of initiators operably associated with said plurality of reservoirs for initiating fluid flow pulsations of said marking fluid from said reservoirs, wherein each initiator of said plurality of initiators is operably associated with a single reservoir of said plurality of reservoirs; a plurality of conduits operably associated with said plurality of initiators, wherein each conduit of said plurality of conduits is operably associated with a single initiator of said plurality of reservoirs; an applicator device comprising a plurality of marking members for applying said marking fluid onto said article, said plurality of marking members operably associated with said plurality of conduits, wherein each of said marking members is operably associated with a single conduit of said plurality of conduits, wherein each nozzle of said plurality of nozzles is operably associated with a single conduit of said plurality of conduits; and a plurality of nozzle tips operably associated with said plurality of nozzles, wherein each nozzle tip of said plurality of nozzle tips is operably associated with a single nozzle of said plurality of nozzles;
activating said initiator to effect fluid flow pulsations of marking fluid from said reservoir through said conduit and to said applicator device; and
applying a plurality of markings to the surface of an article during formation of said article.

11. The method of claim 10 wherein said article is a flexible polymeric hose.

12. The method of claim 10 wherein said plurality of distinctive markings applied to said article is one or more distinctive colors or distinctive patterns.

13. The method of claim 10 wherein said plurality of distinctive markings applied to said article is one or more distinctive colors and one or more distinctive patterns.

14. The method of claim 12 wherein said plurality of distinctive markings is a plurality of distinctive colors.

15. The method of claim 12 wherein said plurality of distinctive markings is a plurality of distinctive patterns.

16. The method of claim 15 wherein said plurality of distinctive patterns are selected from the group consisting of numerals, geometrical designs and text.

17. The method of claim 10 wherein each reservoir of said plurality of reservoirs contains a supply of distinctive colors therein for marking an article.

18. The method of claim 10 wherein said applicator device is a stamping head comprising:
a plurality of marking heads, each of which is operably associated with a single conduit, each of said marking heads having an orifice through which said marking fluid flows from said associated reservoir through said associated conduit to said marking head upon activation of said initiator;
a securing device for securing said stamping head to said apparatus; and
an activator for forcing said stamping head to impinge upon the outer surface of an article for stamping a marking onto the surface of the article to be marked.

19. The method of claim 10 wherein said applicator device is a printing head comprising:
a plurality of nozzles, each of which is associated with a single conduit through which said marking fluid flows from said associated reservoir through said associated conduit to said nozzle upon activation of said initiator;
a securing device for securing said plurality of nozzles to said apparatus; and
a plurality of nozzle tips, each of said plurality of nozzle tips having an orifice therein for ejecting minute volumes of marking fluid onto the surface of the article to be marked.

20. In the manufacture of a flexible polymeric hose having a distinctive mark formed on the surface of said hose during the manufacture of the hose, wherein said distinctive mark represent an indicator for assembly location, the improvement which comprises: simultaneously applying a plurality of distinctive marks on the surface of said hose during finishing operations, wherein said plurality of marks are applied to said flexible polymeric hose by employing a method which comprises:
forming said flexible polymeric hose;
placing a marking apparatus in close proximity to said being formed, said marking apparatus comprising: a plurality of reservoirs having a plurality of marking fluids stored therein, wherein each reservoir of said plurality of reservoirs has stored therein a single marking fluid which is distinctive from any other color stored in any other reservoir of said plurality of reservoirs; a plurality of initiators operably associated with said plurality of reservoirs for initiating flow of said marking fluid from said reservoirs, wherein each initiator of said plurality of initiators is operably associated with a single reservoir of said plurality of reservoirs; a plurality of conduits operably associated with said plurality of initiators, wherein each conduit of said plurality of conduits is operably associated with a single initiator of said plurality of reservoirs; an applicator device comprising a plurality of marking members for applying said marking fluid onto said article, said plurality of marking members operably associated with said plurality of conduits, wherein each of said marking members is operably associated with a single conduit of said plurality of conduits, wherein each nozzle of said plurality of nozzles is operably associated with a single conduit of said plurality of conduits; and a plurality of nozzle tips operably associated with said plurality of nozzles, wherein each nozzle tip of said plurality of nozzle tips is operably associated with a single nozzle of said plurality of nozzles;
activating said initiator by pneumatic pressure or gravity to effect fluid flow pulsations of said marking fluid from said reservoir through said conduit and to said applicator device; and
applying a plurality of markings to the surface of said flexible polymeric hose during a finishing operation.
